(19) United States
(12) Patent Application Publication
(10) Pub. No.: US 2010/0247600 A1
(43) Pub. Date: Sep. 30, 2010

(54) THERAPEUTIC DRUG ELUTING IMPLANT COVER AND METHOD OF MAKING THE SAME

(75) Inventors: Jusong Xia, Collierville, TN (US); Hai H. Trieu, Cordova, TN (US); William F. McKay, Memphis, TN (US); Newton H. Metcalf, JR., Memphis, TN (US); Nikolas F. Kerr, Germantown, TN (US); Drew Amery, Jacksonvill, FL (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/610,005

(22) Filed: Oct. 30, 2009

Related U.S. Application Data
(63) Continuation-in-part of application No. 12/409,899, filed on Mar. 24, 2009.

(51) Int. Cl. A61K 9/00; A61K 47/32; A61K 47/30; A61K 47/42; A61P 31/00; A61K 47/38; A61K 31/65; A61K 31/496

(52) U.S. Cl. 424/423; 514/772.4; 514/772.3; 514/774; 514/773; 514/777; 514/781; 514/154; 514/254.11

(57) ABSTRACT

A drug-eluting implant cover fabricated from a drug-eluting biocompatible matrix containing at least one elutable drug, a drug-eluting implant cover kit containing at least one drug-eluting implant cover, and a method of manufacturing the same.

### CROSS-REFERENCE TO RELATED APPLICATIONS

This application is a continuation-in-part of U.S. patent application Ser. No. 12/409,899, filed Mar. 24, 2009, the entirety of which is incorporated by reference.

This application is related to U.S. patent application Ser. No. ______, filed concurrently herewith, the entirety of which is incorporated by reference.

### BACKGROUND OF THE INVENTION

This invention relates generally to drug-eluting or drug-diffusing implants. More particularly, the invention is for a drug-eluting implant cover having such capability.

Numerous orthopedic implants, including spinal implants such as anterior spinal plates, are known to possess adherent coatings, layers or films containing one or more drugs, e.g., medicaments, therapeutics, biologicals or other bioactive substances, etc., such as antimicrobials, antibacterials, antibiotics, antifungicides, anti-inflammatories, and the like. Following the installation of such an implant in the body, the drug(s) present in the coating elutes therefrom over time into the region of surrounding tissue to achieve the desired drug actions(s).

However, these coatings, layers or films containing one or more drugs are in the form of a non-removable coating that is applied to the implant at the time of manufacture under special manufacturing conditions. One problem that is encountered in the manufacture of implants possessing a drug-eluting coating involves the sterilization of such a device. The more economical methods of sterilization utilize steam under pressure, e.g., as produced in an autoclave. While such sterilization methods are known to be highly effective, they are subject to a major disadvantage where thermally sensitive drugs are concerned and therefore are of limited use. While the conventional use of sterilizing radiation or a sterilant gas such as ethylene oxide can reduce the risk of damaging or partially to completely inactivating the drug component(s) present in the coating component of an orthopedic implant, such sterilization methods are relatively expensive. While it is possible in principle to apply a drug-containing coating to a pre-sterilized implant under sterile conditions followed by the sterile packaging of the coated implant, such an approach to providing a packaged sterile orthopedic implant which avoids subjecting the drug(s) contained in its drug-eluting coating to thermal decomposition or deactivation is largely an impractical one.

In addition, once the coating containing comprising the eluting drug is applied to the implant the eluting drug cannot be changed by the surgeon according to the particular conditions at the time of surgery. In order to provide the surgeon with different concentrations and/or drug compositions in the coating of an implant, several different pre-coated implants must be available in the operating room at the time of surgery and often these implants must be prepped for implant. Part of the prepping procedure is to remove the implant from the sterile enclosure in order to wash it with physiogical solution and have it ready for the surgeon upon request. Once removed from the package, whether used or not, the implant would have to be resterilized or discarded. Since, as stated above, the coating is already on the implant it is difficult to sterilize the unused implants without assuring that the eluting drug is still effective. For these reasons, providing several implants having different drugs/concentrations is often not done since the cost of discarding the implants is prohibitive.

Therefore, what is needed is a drug-eluting cover that can be applied to an implant in vivo or in vitro that can be sterilized separately from the implant and configured to fit a variety of implants is needed. The present invention provides such cover and is described in further detail below.

### SUMMARY OF THE INVENTION

In accordance with the present invention, there is provided a drug-eluting implant cover comprising a pre-formed snap-to-fit implant cover fabricated from a drug-eluting biocompatible matrix comprising at least one elutable drug wherein the drug-eluting biocompatible matrix is configured for therapeutically delivering the at least one elutable drug to a surgical area to facilitate non-irritating motion across adjacent tissue. The implant cover can also be made of or sprayed with a friction-reducing composition in order to reduce irritation of adjacent tissue by the implant.

In accordance with the present invention, there is provided a drug-eluting implant cover kit comprising at least one snap-to-fit implant cover in a sterile container. Kits that fall within the scope of the present invention can also include multiple implant covers having the same or different sizes, having the same or different drugs impregnated in the covers and/or having the same or different concentrations of the elutable drugs impregnated in the cover, all or part of which may be in a sterile container.

According to a further aspect of the present invention, there is provided a method of manufacturing a drug-eluting implant cover of Claim 1, the method comprising forming a drug-eluting biocompatible matrix from a silicone elastomeric material into a pre-determined shape configured to fit an implant to produce a snap-to-fit preformed drug-eluting implant cover; providing a solution comprising a protein synthesis inhibition antibiotic and an RNA polymerase inhibitor antibiotic; immersing said snap-to-fit preformed drug-eluting implant cover in said solution; removing said snap-to-fit preformed drug-eluting implant cover in said solution from said solution; and drying said snap-to-fit preformed drug-eluting implant cover to produce a preformed snap-to-fit drug-eluting implant cover configured to fit a pre-determined shape.

In accordance with the present invention, drug-eluting implant cover of the invention may be made from hydrogels or silicone impregnated with antibacterial/antimicrobial agents such as rifampin, clindamycin and/or minocycline. The present invention may be made from matrix materials, such as silicone, impregnated with antibacterial/antimicrobial agents such as clindamycin, e.g., at weight percent of between about 0.02% and about 0.3%, between about 0.09% and about 0.3%, between about 0.1% and about 0.2% or about 0.15% and rifampin, e.g., at weight percent of between about 0.01% and about 0.1%, between about 0.04% and about 0.07%, between about 0.05% and about 0.06%, or about 0.054%. The present invention can also be made from matrix materials, such as silicone, impregnated with antibacterial/antimicrobial agents such as minocyclin, e.g., at weight percent of between about 0.02% and about 0.8%, between about 0.09% and about 0.3%, between about 0.1% and about 0.2% or about 0.2% and rifampin, e.g., at weight percent of between about 0.03% and about 1.0%, between about 0.09% and about 0.5%, between about 0.1% and about 0.4%, or about 0.3%.

In accordance with the present invention, there is provided a preformed drug-eluding implant cover wherein clindamycin is present in the drug-eluting biocompatible matrix as about 0.15 wt % of the matrix and rifampin is present in the drug-eluting biocompatible matrix as about 0.05 wt % of the matrix.

In accordance with the present invention, there is provided a preformed drug-eluding implant cover wherein rifampin is present in the drug-eluting biocompatible matrix as about 0.3 wt % of the matrix and minocycline is present in the drug-eluting biocompatible matrix as about 0.2 wt % of the matrix.

In accordance with the present invention, there is provided a preformed drug-eluding implant cover wherein the thickness of the drug-eluting device is between about 0.1 mm to about 7 mm, between about 0.2 mm to about 4 mm, between about 0.1 mm to about 2.5 mm, between about 0.1 mm to about 2 mm, between about 0.1 mm to about 1 mm, or between about 0.3 mm to about 1 mm.

According to a further aspect of the present invention, there is provided an implant kit comprising an implant device and at least one drug-eluding implant cover. According to another exemplary embodiment, the invention provides an implant kit comprising at least one preformed drug-eluding implant cover in a package, wherein the at least one preformed drug-eluting implant cover has been sterilized inside the packaging. According to yet another exemplary embodiment, the invention provides an implant kit comprising at least one sterilized preformed drug-eluting implant cover in a packaging, wherein the at least one preformed drug-eluting implant cover is configured to mate with a predetermined implant and cover soft tissue exposed surfaces thereon, thereby delivering the antimicrobial drugs to the soft tissue area and also reducing mechanical irritation during motion by the patient.

In accordance with the present invention, there is provided a method of manufacturing a preformed drug-eluting implant cover comprising forming a drug-eluting biocompatible matrix from a silicone elastomeric material into a predetermined shape configured to securely mate with a predetermined implant device, such as a rod or screw head; creating a solution comprising a protein synthesis inhibition antibiotic and an RNA polymerase inhibitor antibiotic, wherein the solution optionally comprises methylene chloride, xylene, and/or chloroform; optionally, the drug solution concentration is between about 0.1 to about 0.8 grams of each antibiotic per deciliter of solution; immersing the drug-eluting biocompatible matrix in the solution for a period of time, for example, between 30 minutes and an hour; removing the drug-eluting biocompatible matrix from the solution; optionally the method may include purging the solvent from the drug-eluting biocompatible matrix with nitrogen; and drying the drug-eluting biocompatible matrix under a vacuum to produce a preformed drug-eluting implant cover. Optionally, the method may further include packaging the drug-eluting implant cover and/or sterilizing the drug-eluting implant cover in a chamber heated with steam, e.g., by autoclaving the drug-eluting implant cover.

The implant and the drug-eluting structure of this invention can be supplied to the orthopedic surgeon as two separately sterilized components, one being the implant which has been sterilized by the economical autoclave method and the other being a preformed drug-eluting cover which has been sterilized separately, optionally by some other method, e.g., the use of sterilizing radiation or gas plasma sterilization, that does not subject the drug(s) present therein to any significant level of decomposition, denaturation or deactivation. The surgeon then has the choice of affixing the drug-eluting cover to the implant just prior to, during or just after installation of the implant in the body as the particular circumstances may require.

Another major advantage of the implant of the present invention is that it can be assembled at the time of installation from a specific implant and a specific drug-eluting cover which can be selected from amongst a variety of such devices, each differing in the nature and/or amounts of the drug(s) contained therein and/or the nature of the drug-eluting composition, or matrix, from which the device is fabricated thereby offering the surgeon considerable flexibility for choosing the optimal implant and the optimal preformed drug-eluting cover or multiple covers for a particular patient's circumstances and needs. It is far more practical to provide such flexibility of choice in the case of an in situ assembled drug-eluting cover as in the present invention than to provide the same number of choices for a pre-coated implant of the prior art.

To illustrate this advantage, consider the case where a surgeon desires to choose from among 5 different sizes, designs or configurations of implants and five different drugs (and drug combinations) to be eluted. In the case of the in situ assembled implant, the surgeon need only have on hand 5 choices of implants and 5 choices of pre-formed drug-eluting covers to meet all contemplated situations totaling 25 different combinations of assembly of the 5 different implants with the 5 different drug-eluting covers. However, it would require at least 25 pre-coated implants plates of the prior art to provide the same total number of choices. Once the sterility of the pre-coated implants has been compromised in the operating room, the unused pre-coated implants would need to be re-sterilized which, as stated above, requires special equipment and may compromise the drug within the drug eluting coating. Therefore, this is often not done and the surgeon is not given these choices.

Since the drug eluting cover can be packaged separately in sterile packages and the uncoated implants can be easily sterilized using ordinary autoclave machinery, the cost to provide these choices to the surgeon becomes economically feasible when using the present invention. In other words, since an uncoated implant can easily be sterilized using common autoclave and the drug-eluting covers can be individually packaged, there will be little or no waste.

The foregoing scenario points to yet another advantage of the invention over the prior art, namely, it presents the surgeon with the opportunity to choose from among all suppliers' implants to which one or more preformed drug-eluting covers may or may not be affixed. The surgeon is therefore not limited to the specific pre-coated offerings of just one or a few suppliers but has as many choices in this regard as the then-current commercial market makes available.

### BRIEF DESCRIPTION OF THE DRAWINGS

Additional features and aspects of the present invention are described in conjunction with figures in the Detailed Description Section below.

FIG. 1 is a perspective view of one embodiment of an implant and a drug-eluting cover of this invention;

FIG. 2 is a side elevation view of one embodiment of a cervical column having several drug-eluting covers;

FIG. 3 is a side elevation view of one embodiment of a cervical column having several drug-eluting covers;

FIG. 4 is a side elevation view of one embodiment of a cervical column having several drug-eluting covers;

FIG. 5 is a side elevation view of one embodiment of a cervical column having several drug-eluting covers;

FIG. 6 shows several side elevation views of cervical columns having different positioning of drug-eluting covers of the present invention;

FIG. 7 shows several side elevation views of cervical columns having different positioning of drug-eluting covers of the present invention and the drug zones that result;

FIG. 8 shows several side elevation views of cervical columns having drug-eluting covers of the present invention at multi-level of the cervical column;

FIG. 9 shows several side elevation views of cervical columns having drug-eluting covers of the present invention at multi-level of the cervical column;

FIG. 10 shows several side elevation views of cervical columns having drug-eluting covers of the present invention at multi-level of the cervical column;

FIGS. 11 and 11b show surgical pictures of an implant positioned in a Rabbit Model used to test the efficiency of nanosilver vs. particular antibiotic combinations provided in the cover on the implants;

FIGS. 12a and 12b show surgical pictures of the implants coated with nanosilver before implantation and explants after 7 days;

FIG. 13a shows a rabbit suture 7 days after implant of a control tissue which shows clear signs of infection. The site of infection is circled and labeled in FIG. 13a. FIG. 13b shows a clean rabbit suture line of a nanosilver coated implant also 7 days after being implanted;

FIG. 14 illustrates biofilm formation with initial attachment of the microbes to the surface of the implant, expansion of the population, maturation of the population within the biofilm, and finally disruption of the biofim and liberation of the microbes into the surrounding tissue where they can again recolonize the implant or migrate elsewhere in the subject (in the experiments, sonication was used to disrupt the biofilm prior to assaying for the presence of microbes);

FIG. 15 is a perspective view of the rod component of a spinal fixation system and a performed drug-eluting sleeve in accordance with this invention about to be affixed to the rod;

FIG. 16 is a perspective view of the rod component of a spinal fixation system and a performed drug-eluting sleeve in accordance with this invention being affixed to the rod with the affixation of the sleeve to the rod being illustrated in the cross sectional views A-D;

FIG. 17 is a perspective view of a drug-eluting cover in the form of a cap configured to fit onto ends, pointed protrusions, screw heads, etc. of an implant; and

FIG. 18 illustrates the effect of sterilization on the final drug concentration, showing the degradation rate versus autoclave time, for example, with a starting concentration of about 0.0738 wt % rifampin in the silicone and running the autoclave about 30 minutes will result in a final concentration of about 0.054 wt % rifampin in the silicone sleeve.

### DETAILED DESCRIPTION OF THE INVENTION

Implants such as orthopedic prosthetic implants constructed of plastics, polymers, metals, ceramics or materials made from composites of these materials to address orthopedic injuries and deformities has become commonplace. Such implants typically have one or more surfaces that are placed in direct contact with living tissues and some implants include surfaces against which living tissues of the host slide or otherwise move in normal use. In this arena, concerns are sometimes raised about decreasing the invasiveness of the implants and the procedures for implanting them, improving implant integrity, and improving patient outcomes.

Despite the many positive benefits that are gained by the use of such implants, contact between the surfaces of the implant and soft tissues of the host, including muscle tissues, blood and the like, can produce unwanted results. For example, dynamic contact between the surfaces of the implant and soft tissue of the host can cause significant abrasive damage to fragile and sensitive human cells and tissues, which may result in increased reporting of pain associated with the implant or prolonged pain following insertion of the device. These dynamic contacts can also cause a wide range of undesirable effects such as tissue and cell adhesion, irritation, inflammation, thrombogenicity (clotting of the blood), hemolysis, unwanted mineral deposits, and increased pain or limited motion, to name a few.

To overcome many of these problems some implants or parts of an implant can be covered with a drug-eluting snap-to-fit cover that covers the surface of a specific implant and not only releases drugs, but is configured to facilitate non-irritating motion across adjacent tissue surfaces such as soft tissue. In one embodiment of the invention, the drug-eluting cover is made of or coated with at least one friction-reducing material to reduce irritating motion across soft tissue.

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems, or devices, and any further applications of the principles of the invention as described herein, are contemplated as would normally occur to one skilled in the art to which the invention relates.

For the purpose of this application the following definitions are provided to aid in the understanding of the invention.

The term “about” shall be understood herein to mean less than or equal to a 15% deviation from the recited value, for example, a rifampin concentration of about 0.054 wt % means rifampin at 0.054 wt %±15% (or, rounding to the same decimal place, between 0.053 wt % and 0.055 wt %).

The term “implant” or “spinal implant” shall be understood herein to include spinal implants of all kinds including spinal stabilization implants, spinal dynamic implants, spinal rods, spinal plates, anterior spinal plates, spinal rod components of a spinal fixation system, spinal fixation systems, spinal interbody fusion devices, bone screws, pedicle screws, crosslink components, spinal hooks, interspinous process spacers, bone rods, etc., and their sub-assembly components thereof, if any, e.g., the components by which they are affixed to bone such as screws, staples, ties, bands, etc.

The term “biocompatible” as applied to the drug-eluting material from which the drug-eluting implant herein is fabricated shall be understood in its ordinary art-recognized sense as describing a material exhibiting a relatively low chronic tissue response for the period that the material is present in the body.

The expression “drug-eluting” shall be understood to refer to any and all mechanisms, e.g., diffusion, migration, permeation, and/or desorption by which the drug(s) incorporated in the coating pass therefrom over time into the surrounding body tissue.

The expression “drug-eluting coating” shall be understood herein to mean any natural, synthetic or semi-synthetic material into which one or more drugs can be incorporated and from which the incorporated drug(s) are capable of eluting over time.

The expression “elutable drug” shall be understood to mean a drug having the ability to pass over time from the drug-eluting coating in which it is incorporated into the surrounding areas of the body.

The term “drug” includes all medically useful bio-affecting and body-treating compositions.

The term “weight percent” or “wt %” means the ratio of the drug weight to the weight of the biocompatible matrix, e.g., the silicone after sterilization, for example, an initial concentration of rifampin of about 0.0738 wt % with about a 30 minute autoclave time will result in a final wt % of about 0.054 in the sterilized product. Other than where expressly indicated, all numbers expressing amounts of materials, concentrations, quantified properties of materials, and so forth, stated in the specification and claims are to be understood as being modified in all instances by the term “about” or “approximately”.

It will also be understood that any numerical range recited herein is intended to include all sub-ranges within that range and any combination of the various endpoints of such ranges or subranges.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

Orthopedic implants of many configurations, sizes and functions and their use in the surgical treatment of bone injuries and defects are well known in the art. The implants can be fabricated from a wide range of materials including metals, synthetic polymers, ceramics and bone. Examples of these materials include metals such as medical grade stainless steel, titanium and titanium alloys, and the like, synthetic polymers such as thermoplastic polymers, thermoset polymers, elastomers, and the like, ceramics such as pyrolytic carbon, carbon fibers, and their composites, zirconia, alumina, titanic and their composites, and the like, bone, e.g., autograft, allograft, xenograft or transgenic cortical and/or corticocancellous bone obtained, e.g., from the femur, tibia, fibula radius and/or ulna and provided as a single unit or as a composite built up from smaller bone elements and/or bone-derived particles. The implants can be fabricated with collagen, demineralized bone matrix (DBM), bone morphogenetic proteins (DMP) and combinations thereof.

An illustrative orthopedic implant, an anterior spinal plate, can come in many different sizes and configurations for installation at various cervical, thoracic and lumbar regions of the spine. Its fastener components (typically screws) aside, an anterior spinal plate can be provided as a single unit or as an assemblage of two or more sub-units. Illustrative spinal plates are those described in, among others, U.S. Pat. Nos. 6,193,721; 6,206,882; 6,224,602; 6,228,085; 6,258,089; 6,342,055; 6,413,259; 6,533,786; 6,602,255; 6,602,256; 6,605,090; 6,613,053; 6,679,883; 6,755,833; 6,761,719; 7,041,105; 7,169,150; 7,186,256; 7,306,605; 7,468,069; and 7,481,829, and U.S. Patent Application Publication Nos. 2004/0204712; 2005/0192577; 2005/0228386; 2007/0043369; 2007/0233110; 2007/0276371; 2008/0234753; 2009/0012571; and 2009/0024171, the entire contents of which are incorporated by reference herein.

Another type of well-known orthopedic implant, a spinal fixation system, typically consists of separate rods, screws, connectors, and in same cases, still other components, for assembly at the surgical site. Illustrative of spinal fixation systems are those described in, among others, U.S. Pat. Nos. 5,334,203; 5,534,002; 5,562,662; 5,609,592; 5,611,800; 5,899,901; 6,050,997; 6,132,430; 6,136,002; 6,280,443; 6,395,030; 6,416,515; 6,562,040; 7,141,051; and 7,314,467, the entire contents of which are incorporated by reference herein.

Coating-containing drug reservoirs that are distinguishable from the present invention have been described in U.S. Patent Application Publication Nos. 2005/0031666; 2007/0299520; 2006/0047341; 2007/0270858; 2004/0030342; and 2007/0173934, the entire contents of which are incorporated by reference herein.

Prior to the surgical installation of a selected orthopedic implant, there is covered at least a portion of the surface of the implant, e.g., a portion of an exposed surface, with a drug-eluting biocompatible implant cover containing one or more elutable drugs in accordance with this invention.

The drug-eluting cover can possess a planar shape, e.g., that of a square, rectangle, circle, oval, etc., which can be wrapped around at least a portion of the implant and affixed in place. In the alternative, the drug eluting cover can be in the form of a sleeve or cap form that can be slipped over the implant and held in place by the natural clinging of the elastic cover to at least a portion of the implant as described herein, or can be affixed by other means including a biological adhesive. Still further the drug-eluting cover can be produced in sheets or long sleeves that can be cut to size by the surgeon at the time of surgery and can either be snapped (elastic) or affixed to an implant. Still further, the drug-eluting cover can also be produced in the form of a cap configured to fit onto ends, pointed protrusions, screw heads, etc. of an implant that might result in irritation of adjacent tissue. Thus, giving the maximum choices to the surgeon.

The matrix can be formed from a material of homogeneous or heterogeneous composition, can possess a single layer or multiple layers (i.e., a laminate), can be rigid, flexible or semiflexible, can be stretchable (elastic) so as to engagedly fit some portion of its associated implant or nonstretchable (inelastic), can be porous or non-porous, can vary considerably in its average dimensions, etc. The drug eluting cover can have at least one extension configured to fit within a complimentary cavity located on the surgical implant. The drug eluting cover can be positioned so that the extension (or extensions) snap (or pressure fit) into or around the surgical implant. In this embodiment, the surface of the drug-eluting matrix is in direct contact with at least one surface of the implant.

Since no contact between surfaces is absolutely perfect, gaps and/or air pockets can and will arise between the drug eluting cover and the surface of the implant. These gaps and/or pockets can develop infections which can be prevented by use of antimicrobial/anti-infectious compounds which elude from the matrix into the pockets that may form. In addition to preventing infection in these gap/pockets, the drug-eluting matrix also eludes into the soft tissue surrounding the implant. The present invention is configured to allow the drug to elute from the cover towards the surface of the implant as well as to the surrounding soft tissue. This feature aids in preventing any infections that may arise between the drug eluting layer and the surface of the implant as well in the surrounding tissue.

The drug-eluting cover can be dimensioned and configured as desired by any suitable technique, e.g., molding, machining, die-cutting from a larger sheet or section, etc., and can be dimensioned and configured by the surgeon or assistant personnel, e.g., by scissors if the nature of the drug-eluting matrix permits, at or near the time the drug-eluting cover is to be used affixed to the selected implant.

The drug-eluting cover or drug-eluting biocompatible matrix can be fabricated from amongst any of the numerous biocompatible materials heretofore known for providing drug-eluting covers. Useful covers include film-forming polymer(s), non-bioresorbable, or non-bioabsorbable, materials and bioresorbable, or bioabsorble, materials. Natural, semi-synthetic and fully synthetic polymers of both types are well known in the art for use as drug-eluting covers.

Film-forming polymer(s) are selected from the group consisting of bioresorbable polymer, hydrogel, polylactide, polyglycolide, copolymers of polylactide, polyglycolide, polycaprolactone, polyorthoester, silicone, polyurethane, silicone-polyurethane copolymers, polyethylene, polypropylene, polyester, polyaryletherketone, polyimide, polyetherimide, polyamide, polysulfone and combinations thereof.

Among the useful non-bioresorbable drug-eluting covers are biocompatible polymers such as polyurethanes, silicones (polysiloxanes), polyesters, polyamides, polyolefins such as polyethylene, polypropylene, polyisobutylene and their copolymers, acrylic polymers and copolymers, vinyl halide polymers and copolymers, fluorocarbon polymers such as polytetra fluoroethylene, polyvinyl ethers, polyacrylonitriles, polyvinyl ketones, polyvinyl aromatics such as polystyrene, polyvinyl esters, polycarbonates, polyimides, polyethers, epoxy resins, compatible blends of these and other biocompatible polymers, and the like.

Useful bioresorbable drug-eluting covers include hydrogels and polymers, such as poly(L-lactic acid), poly(glycolic acid), poly(lactide-co-glycolide), polydioxanone, polyorthoesters, polyanhydrides, and the like. For example, such polymers include but are not limited to a polyvinyl alcohol, a polyacrylic acid, a polyarylamide, a poly(acrylonitrile-acrylic acid), a polyurethane, a polyethylene glycol, a poly(N-vinyl-2-pyrrolidone), a gelatin, a collagen, a polysaccharide, a cellulose, and combinations thereof. The hyrdogels of the present invention may be hydrated or unhydrated. The unhydrated hydrogels of the present invention will become hydrated either prior, during or after implantation. Once hydrated the hydrogels will increase in dimensions.

Hydrogels that can be used for the present invention may also be made non-bioresorbable by means of the process in which they are produced as well as the molecular composition. Various degrees of bioresorbability can also be accomplished by varying the amount of crosslinking in the hydrogel. Useful hydrogels are selected from the group consisting of a polyvinyl alcohol, a polyacrylic acid, a polyarylamide, a poly(acrylonitrile-acrylic acid), a polyurethane, a polyethylene glycol, a poly(N-vinyl-2-pyrrolidone), a gelatin, a collagen, a polysaccharide, a cellulose, and combinations thereof.

If desired, the drug-eluting cover herein can be provided as a laminate with, e.g., a first layer (the layer closest to the surface of the implant to which the cover will be placed) fabricated from a non-bioresorbable cover containing one or more elutable drugs and superimposed thereon a second layer of bioresorbable cover containing the same or different drug(s) as the first layer and/or a lubricious material to reduce adjacent tissue irritation.

The drug-eluting properties of a drug-eluting cover, principally the rate of release of its drug component(s) into the surrounding body tissues, is of prime importance. Those skilled in the art employing known procedures can readily select the optimum drug-eluting cover material for a particular drug or drug combination and drug loading(s).

The selected drug(s) can be incorporated in the drug-eluting cover during and/or after the formation of the drug-eluting cover material. The incorporation of drug can be substantially uniform or the drug(s) can be distributed in the drug-eluting cover in gradient fashion or in distinct zones of concentration employing any of several methods known in the art. Thus, e.g., a greater concentration of drug(s) at or near the exposed surface of the drug-eluting cover can be made to provide an initially higher concentration of drug(s) in the surrounding tissues followed by a reduction in delivered drug concentration (and perhaps longer term drug delivery as well if desired) as the more interior regions or zones of lower drug concentration within the drug-eluting cover begin eluting the drug. This gradient or zonal distribution of drug in the drug-eluting cover can be utilized to initially deliver a higher concentration of one drug in a drug combination followed by later delivery of a higher concentration of another drug in the drug combination.

Useful drug incorporation procedures include combining the selected elutable drug(s) with the precursor(s) of the cover and thereafter forming the cover. Thus, in the case of a polymeric cover, e.g., an open cell polyurethane foam, the drug(s) can be admixed with the precursor reactants (e.g., polyisocyanate and polyol among other components of the polyurethane foam-forming reaction mixture) with the resulting polyurethane foam entraining the drug(s).

Another drug incorporation procedure involves contacting the drug-eluting cover material with a drug-containing solvent medium which dissolves the cover and, following evaporation of the solvent(s), leaves the drug(s) in the reconstituted cover. A similar procedure involves contacting the drug-eluting cover with a drug-containing swelling agent and allowing the drug(s) to diffuse into the cover.

When an open cell cover is used as the drug-eluting vehicle, e.g., the aforementioned polyurethane foam, the desired drug(s) can be incorporated in the cover by immersion in a suitable aqueous and/or organic solvent solution of the drug(s) followed by draining excess solvent and if desired, drying.

The drug-eluting cover can also be fashioned from organic and/or inorganic particulate material and drug bonded together in the desired configuration employing a biocompatible bonding or binder material. Examples of a binder material include the resorbable or non-resorbable biomaterials mentioned above. Additional examples of binder materials include those used in the pharmaceutical industry such as polysaccharides, celluloses, collagen material, gelatin material, synthetic bioresorbable polymer, etc.

These and/or other known techniques can also be used to incorporate one or more non-drug materials in the cover. Among some optional non-drug materials that can be incorporated in the drug-eluting cover or are used to coat the drug-eluting cover are diluents, carriers, excipients, stabilizers, permeation enhancers, surface active agents, anti-adhesion agents and the like, in known and conventional amounts.

The amounts of elutable drug for incorporation in the drug-eluting cover herein will depend on a number of factors well understood by those skilled in the art including the nature of the selected drug(s), the nature, amounts and configuration of the selected cover and the desired profile (rate and duration) of drug release into the surrounding tissues. Again, empirical investigation employing known and conventional procedures can be utilized by those skilled in the art to arrive at an optimum concentration of specific drug(s) for a specific cover. The concentration of drug(s) and the drug-eluting profile of the drug-eluting implant cover will be such as to deliver a therapeutically effective concentration of the desired drug(s) for a therapeutically useful duration. Total concentration of deliverable drug can range, e.g., from 0.03 to 2, or from 0.01 to 3, weight percent of the drug-eluting cover and can provide eluted drug(s) in therapeutically useful amounts for periods ranging e.g., for at least 24 hours and preferably at least 70, 100, 250, 500 or even 750 hours or more. In certain embodiments, the duration of effective drug release can range from 1 to 3 weeks. In an exemplary embodiment, the drug-eluting implant cover may be made from a matrix material, such as silicone, and comprise a combination antibacterial/antimicrobial agents such as clindamycin, e.g., at weight percent of between about 0.02% and about 0.3%, between about 0.09% and about 0.3%, between about 0.1% and about 0.2% or about 0.15% and rifampin, e.g., at weight percent of between about 0.01% and about 0.1%, between about 0.04% and about 0.07%, between about 0.05% and about 0.06%, or about 0.054%. In another exemplary embodiment, the drug-eluting implant cover may be made from matrix materials, such as silicone, and impregnated with antibacterial/antimicrobial agents such as minocyclin, e.g., at weight percent of between about 0.02% and about 0.8%, between about 0.09% and about 0.3%, between about 0.1% and about 0.2% or about 0.2% and rifampin, e.g., at weight percent of between about 0.03% and about 1.0%, between about 0.09% and about 0.5%, between about 0.1% and about 0.4%, or about 0.3%.

As previously indicated, the dimensions of the drug-eluting cover can vary considerably. Thus, the surface dimensions of the cover can be such as to exceed, match or be less than that of the surface dimensions of the orthopedic implant to which it is covered. By way of illustration, in the case of an anterior cervical plate having an average major surface dimension (e.g., length) of 25 mm and a minor surface dimension (e.g., width) of 12 mm, the drug-eluting implant to be covered thereto can possess a length of from 5 to 27 mm and a width of from 2 to 14 mm.

The thickness of the drug-eluting cover can influence the rate of drug release from the implant and can vary considerably depending on the drug release profile desired. In one embodiment, the thickness of the drug-eluting implant cover is between about 0.1 mm to about 7 mm, between about 0.2 mm to about 4 mm, between about 0.1 mm to about 2.5 mm, between about 0.1 mm to about 2 mm, between about 0.1 mm to about 1 mm, or between about 0.3 mm to about 1 mm.

The drug(s) selected for incorporation in the drug-eluting implant cover can in their essentially pure and/or concentrated form be a solid material, e.g., a powder, a semi-solid or a liquid of widely varying appearance. The physical properties and characteristic elution rates from a given drug-eluting cover can be determined by a person of ordinary skill in the art, including when the drug is encased in a dissolvable solid bead or liposome for delayed release. When desired, a drug can be incorporated in the drug-eluting cover in both an encapsulated form and a free form via suitable carrier liquids, e.g., solvents, in particular, water, organic solvent(s) or aqueous mixtures of organic solvent(s). In addition, the drug-eluting cover may optionally contain one or more non-drug materials, e.g., one or more of those previously recited, dissolved, suspended or dispersed therein. It will, of course, be appreciated that when the physical form of the pure and/or concentrated drug is that of a solid or semi-solid, it may be beneficial if at least some portion of the carrier with the drug(s) dissolved, suspended or dispersed therein is retained in the cover for subsequent delivery of such drug(s) to the surrounding region of tissue.

The drug(s) or elutable drug(s) incorporated in the drug-eluting cover herein include, inter alia, antibiotic agents, antiseptic agents, antiviral agents, analgesics, bone growth promoting substances, anti-inflammatants, anti-arrhythmics, anti-coagulants, antifungal agents, growth inhibitors, growth stimulators, steroids, anti-adhesion agents, growth factor agents, wound-healing accelerators, immuno-suppressants, bone morphogenic proteins, soft tissue growth inhibitors, local anesthetics and/or any of numerous other classes of therapeutic agents.

Any antibiotic suitable for use in a human may be used in accordance with various embodiments of the invention. As used herein, “antibiotic” means an antibacterial agent. The antibacterial agent may have bateriostatic and/or bacteriocidal activities. Nonlimiting examples of classes of antibiotics that may be used include tetracyclines (e.g. minocycline), rifamycins (e.g. rifampin), lincosamides (e.g. clindamycin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sulfonamides (e.g. sulfamethoxazole), glycopeptides (e.g. vancomycin), quinolones (e.g. ciprofloxacin), fusidic acid, trimethoprim, metronidazole, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, clindamycin, erythromycin, nafcillin, cefazolin, imipenem, aztreonam, gentamicin, sulfamethoxazole, vancomycin, ciprofloxacin, trimethoprim, metronidazole, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, listed in U.S. Pat. No. 4,642,104, the entire contents of which are incorporated by reference herein, may also be used. One of ordinary skill in the art will recognize still other antibiotics that may be used.

In general, it is desirable that the selected antibiotic(s) kill or inhibit the growth of one or more bacteria that are associated with infection following surgical implantation of a medical implant. Such bacteria are recognized by those of ordinary skill in the art and include Staphylococcus aureus, Staphylococcus epidermis, Staphylococcus capitis, Escherichia coli, and Acinetobacter baummanii. Preferably, the antibiotic(s) selected are effective against strains of bacteria that are resistant to one or more antibiotic.

To enhance the likelihood that bacteria will be killed or inhibited, it may be desirable to combine two or more antibiotics. It may also be desirable to combine one or more antibiotics with one or more antiseptics. It will be recognized by one of ordinary skill in the art that using two or more antimicrobial agents having different mechanisms of action and/or different spectrums of action may be most effective in achieving the desired effect. In one embodiment, the elutable drug is rifampin in combination with minocycline and/or clindamycin.

Any antiseptic suitable for use in a human may be used in accordance with various embodiments of the invention. As used herein, the term “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver-containing compounds (silver sulfadiazene, silver metal, silver ion, silver nitrate, silver acetate, silver protein, silver lactate, silver picrate, silver sulfate), and alcohols. One of ordinary skill in the art will recognize other antiseptics that may be employed in accordance with this disclosure.

It is desirable that the selected antiseptic(s) kill or inhibit the growth of one or more microbial species that are associated with infection following surgical implantation of a medical implant. Such microbes are recognized by those of ordinary skill in the art and include Staphylococcus aureus, Staphylococcus epidermis, Escherichia coli, Pseudomonus aeruginosa, and Candida.

To enhance the likelihood that microbes will be killed or inhibited, it may be desirable to combine two or more antiseptics. It may also be desirable to combine one or more antiseptics with one or more antibiotics. It will be recognized by one of ordinary skill in the art that antimicrobial agents having different mechanisms of action and/or different spectrums of action may be most effective in achieving the desired effect of inhibiting a broad spectrum of potentially infectious microbes and/or drug resistant microbes. In a particular embodiment, a combination of chlorohexidine and silver sulfadiazine is used.

Any antiviral agent suitable for use in a human may be used in accordance with various embodiments of the invention. Nonlimiting examples of antiviral agents include acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. One of ordinary skill in the art will recognize other antiviral agents that may be employed in accordance with this invention.

To enhance the likelihood that viruses will be killed or inhibited, it may be desirable to combine two or more antiviral agents. It may also be desirable to combine one or more antiseptics with one or more antiviral agent.

Any anti-fungal agent suitable for use in a human may be used in accordance with various embodiments of the invention. Nonlimiting examples of anti-fungal agents include amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione. One of ordinary skill in the art will recognize other anti-fungal agents that may be employed in accordance with this disclosure.

To enhance the likelihood that viruses will be killed or inhibited, it may be desirable to combine two or more anti-fungal agents. It may also be desirable to combine one or more antiseptics with one or more anti-fungal agent.

Any anti-inflammatory agent suitable for use in a human may be used in accordance with various embodiments of the invention. Non-limiting examples of anti-inflammatory agents include steroids, such as cortisone, hydrocortisone, prednisone, dexamethasone, methyl-prednisilone, an, derivatives thereof; and non-steroidal anti-inflammatory agents (NSAIDs). Non-limiting examples of NSAIDS include ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

Non-limiting examples of other pharmacological agents that may be used include: beta-radiation emitting isotopes, beclomethasone, fluorometholone, tranilast, ketoprofen, curcumin, cyclosporin A, deoxyspergualin, FK506, sulindac, myriocin, 2-aminochromone (U-86983), colchicines, pentosan, antisense oligonucleotides, mycophenolic acid, etoposide, actinomycin D, camptothecin, carmustine, methotrexate, adriamycin, mitomycin, cis-platinum, mitosis inhibitors, vinca alkaloids, tissue growth factor inhibitors, platinum compounds, cytotoxic inhibitors, alkylating agents, antimetabolite agents, tacrolimus, azathioprine, recombinant or monoclonal antibodies to interleukins, T-cells, B-cells, and receptors, bisantrene, retinoic acid, tamoxifen, compounds containing silver, doxorubicin, azacytidine, homoharringtonine, selenium compounds, superoxide-dismutase, interferons, heparin; antineoplastic/antiangiogenic agents, such as antimetabolite agents, alkylating agents, cytotoxic antibiotics, vinca alkaloids, mitosis inhibitors, platinum compounds, tissue growth factor inhibitors, cisplatin and etoposide; immunosuppressant agents, such as cyclosporine A, mycophenolic acid, tacrolimus, rapamycin, rapamycin analogue (ABT-578) produced by Abbott Laboratories, azathioprine, recombinant or monoclonal antibodies to interleukins, T-cells, B-cells and/or their receptors; anticoagulents, such as heparin and chondroiten sulfate; platelet inhibitors such as ticlopidine; vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol; thrombolytic agents, such as stretokinase, urokinase and tissue plasminogin activators; analgesics and antipyretics, such as the opioid analgesics such as buprenorphine, dextromoramide, dextropropoxyphene, fentanyl, alfentanil, sufentanil, hydromorphone, methadone, morphine, oxycodone, papavereturn, pentazocine, pethidine, phenopefidine, codeine dihydrocodeine; acetylsalicylic acid (aspirin), paracetamol, and phenazone; and, antiproliferative agents such as QP-2 (taxol), paclitaxel, rapamycin, tacrolimus, everolimus, actinomycin, methotrexate, angiopeptin, vincristine, mitocycin, statins, C-MYC antisense, sirolimus, restenASE, 2-chloro-deoxyadenosine, PCNA (proliferating cell nuclear antigent) ribozyme, batimastat, prolyl hydroxylase inhibitors, halofuginone, C-proteinase inhibitors, and probucol; and combinations and/or derivatives thereof.

The drug-eluting cover can be made of or coated with a lubricious friction-reducing formulation and/or anti-adhesion agent to reduce irritating motion across adjacent tissue. Suitable friction-reducing formulations comprise silicone, hydrogel, xerogel, polyethylene, lotions, lubricants, oils, greases, fluoro-polymer, hydrophilic agents, and combinations thereof.

A suitable anti-adhesion agent(s) is a chemical or physical agent that forms a barrier on a surface of a cover on a medical device such as an implant and through the absence of cohesive strength and/or weak boundary layers, reduces or prevents adhesion of that surface of the cover to a material such as, but not limited to, another portion of the cover or an uncovered portion of the medical device. Examples of suitable physical anti-adhesion agents include, without limitation, solid glass spheres, glass bubbles, other mineral, or polymeric particles. Suitable anti-adhesion agents include any surface active compositions which reduces the surface tack of the cover. These agents may be known polymeric anti-adhesion agents such as silicones and fluorine containing polymers, for example. These agents may also consist of known biosorbable and biodegradable compositions which act to reduce the surface adhesive properties. These agents may further include intermediate molecular weight compounds such as oligomers of polyethers and alkanes, or biological oils such as fatty esters, to name a few. These agents may also be low molecular weight surface active compounds such as low molecular weight silicones, fluorinated materials, or biological compounds such as sugars.

Chemical anti-adhesion agents may further include various surfactant compositions which may be nonionic or ionic in composition. Nonionic surfactants are defined as those agents which are amphiphilic in nature but do not readily ionize in aqueous solution. Nonionic surfactants may include, for example C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, clyceryl monolaurate, glyceryl mondocosanoate, glyceryl monomyristate, glyceryl monodicenoate, glyceryl dipalmitate, glyeryl didocosanoate, glyceryl dimyristate, glyceryl didecenoate, glyceryl tridocosanoate, glyceryl trimyristate, glyceryl tridecenoate, glycerol tristearate and mixtures thereof, sucrose fatty acid esters such as sucrose distearate and sucrose palmitate; sorbitan fatty acid esters such as sorbitan monostearate, sorbitan monopalmitate and sorbitan tristerate; C₁₆-C₁₈ fatty alcohols such as cetyl alcohol, myristyl alcohol, stearyl alcohol, and cetostearyl alcohol, esters of fatty alcohols or fatty acids such as cetyl palmitate and cetearyl palmitate; anhydrides of fatty acids such as stearic anhydride. Nonionic surfactants may further include various metallic salts, such as calcium stearate, magnesium stearate, and zinc stearate, to name a few. Nonionic surfactants may also include organo-onium compounds. Ionic surfactants are defined as those agents which are polar in nature and readily ionize in solution. Ionic surfactants would generally include organic compounds containing salts of strong acid and bases. Examples of ionic surfactants would include, for example, lauryl sulfates such as ammonium lauryl sulfate. Ionic surfactants may further include certain biological lipids, such as phosphatidyl coline. The chemical or physical anti-adhesion agent can be present in the drug-eluting cover or in a separate cover(s) in an amount to achieve the desired level of anti-adhesion.

The drug-eluting implant cover kit of the present invention comprises at least one snap-to-fit implant cover in a sterile container. The kit can provide multiple snap-to-fit implant covers of the same or a different size in a sterile container, wherein at least two implant covers have elutable drugs in different concentrations. Further, the implant cover(s) in the kit can can be configured to fit at least a part of an implant selected from the group of a spinal stabilization implant, spinal dynamic implant, spinal rod, spinal plate, anterior spinal plate, spinal rod component of a spinal fixation system, spinal interbody fusion device, bone screw, pedicle screw, crosslink component, spinal hook, interspinous process spacer, assembly or sub-assembly thereof. In addition, the at least one elutable drug in the kit can include a combination of minocycline and rifampin or a combination of clindamycin and rifampin. The amount of the elutable drugs in the cover of the implant(s) provided in the kit can comprise minocycline at a level of from a weight percent of between about 0.02% and about 0.8%, between about 0.09% and about 0.3%, between about 0.1% and about 0.2% or about 0.2% and rifampin, e.g., at weight percent of between about 0.03% and about 1.0%, between about 0.09% and about 0.5%, between about 0.1% and about 0.4%, or about 0.3%. and/or clindamycin at a level of from about 0.02% to about 0.3%, at a level of from about 0.09% to about 0.3%, at a level of from about 0.1% to about 0.2% or at a level of about 0.15%, weight percent, the drug-eluting biocompatible cover in combination with rifampin at a level of from a weight percent of between about 0.01% and about 0.1%, between about 0.04% and about 0.07%, between about 0.05% and about 0.06%, or about 0.054% of the drug-eluting biocompatible matrix material.

Provided is a method for surgically installing a drug-eluting spinal implant in a body which comprises surgically installing a spinal implant at least partially covered with a drug-eluting biocompatible cover comprising at least one elutable drug wherein said drug-eluting biocompatible cover is configured for therapeutically delivering said at least one elutable drug to a surgical area in the body of a patient and to facilitate non-irritating motion across soft tissue. The method of surgically installing the drug-eluting spinal implant provides for an implant comprising minocycline at a level described herein and/or clindamycin at a level at a level described herein in combination with rifampin at a level described herein.

Also provided is a method of manufacturing a drug-eluting implant cover of the present invention. The method comprises forming a drug-eluting biocompatible matrix from a silicone elastomeric material into a pre-determined shape configured to fit an implant to produce a snap-to-fit preformed drug-eluting implant cover, providing a solution comprising a protein synthesis inhibition antibiotic and an RNA polymerase inhibitor antibiotic; immersing said snap-to-fit preformed drug-eluting implant cover in said solution; removing said snap-to-fit preformed drug-eluting implant cover in said solution from said solution; and drying said snap-to-fit preformed drug-eluting implant cover to produce a preformed snap-to-fit drug-eluting implant cover configured to fit a pre-determined shape. The method can further include a coating step comprising dipping said pre-formed snap-to-fit drug eluting implant cover configured to fit a pre-determined shape into a lubricious friction reducing formulation or spraying said lubricious friction reducing formulation onto said pre-formed snap-to-fit drug eluting implant cover configured to fit a pre-determined shape. Further, the drug-eluting implant cover can be configured to fit at least a part of an implant selected from the group of a spinal stabilization implant, spinal dynamic implant, spinal rod, spinal plate, anterior spinal plate, spinal rod component of a spinal fixation system, spinal fixation system, spinal interbody fusion device, bone screw, pedicle screw, crosslink component, spinal hook, interspinous process spacer, bone rod and sub-assembly components thereof. The coating step can be carried out prior to or during installing of said implant.

In another exemplary embodiment, there is provided a method of manufacturing a preformed drug-eluting implant cover comprising forming a drug-eluting biocompatible matrix from a silicone elastomeric material into a pre-formed shape configured to snap-to-fit onto a predetermined medical implant or part of an implant; creating a solution comprising a protein synthesis inhibition antibiotic and an RNA polymerase inhibitor antibiotic, wherein the solution optionally comprises methylene chloride, xylene, and/or chloroform; optionally, the drug solution concentration is between about 0.1 to about 0.8 grams of each antibiotic per deciliter of solution; immersing the a pre-formed implant cover in the solution for a period of time, for example, between 30 minutes and an hour; removing the a pre-formed implant cover from the solution; optionally the method may include purging the a pre-formed implant cover with nitrogen; and drying the a pre-formed implant cover under a vacuum to produce a preformed snap-to-fit drug-eluting implant cover. Optionally, the method may further include packaging the drug-eluting implant cover and/or sterilizing the drug-eluting implant cover in a chamber heated with steam, e.g., by autoclaving the drug-eluting implant cover.

In one embodiment of the present invention, the drug-eluting cover is made of a silicone elastomer used for spinal rods, wherein the implant cover contains Rifampin and Minocycline or Rifampin and Clindamycin.

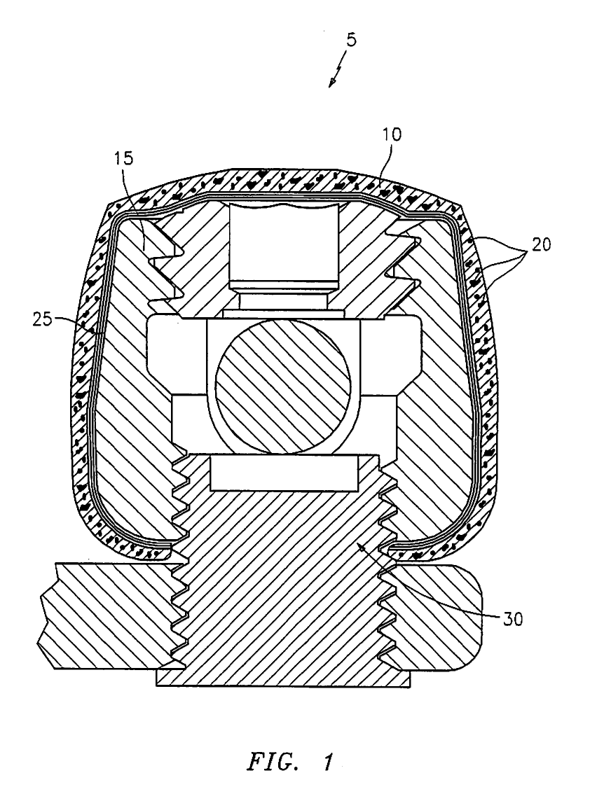

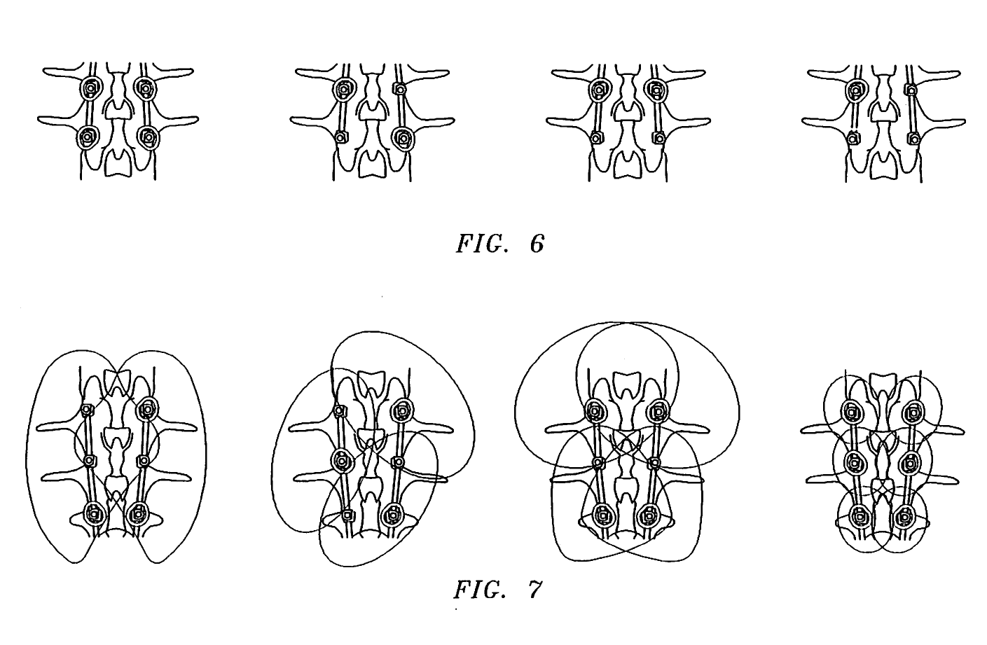

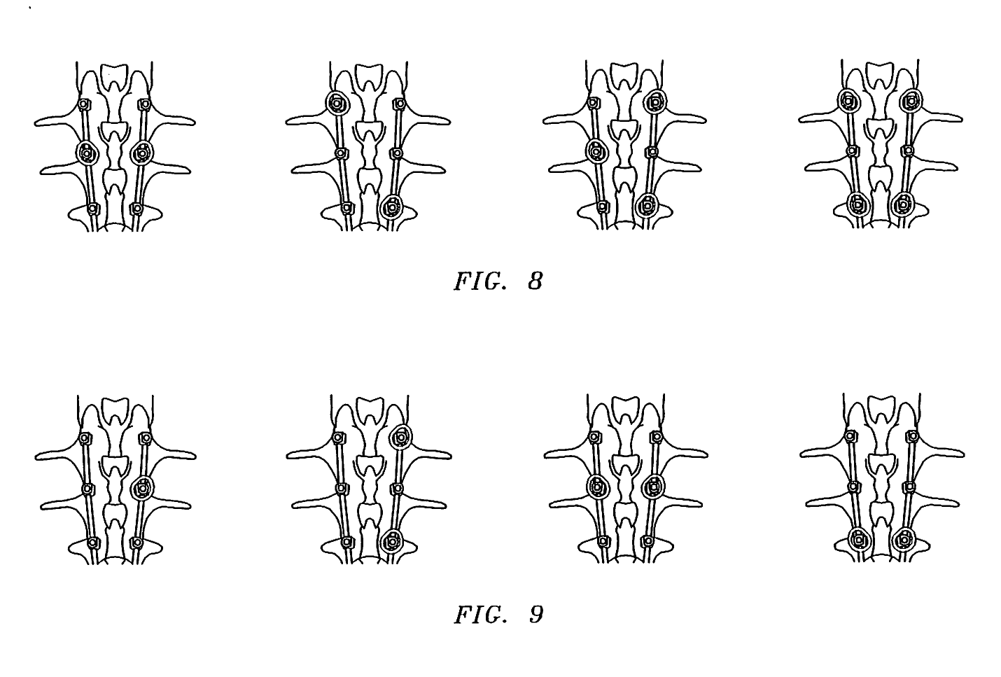

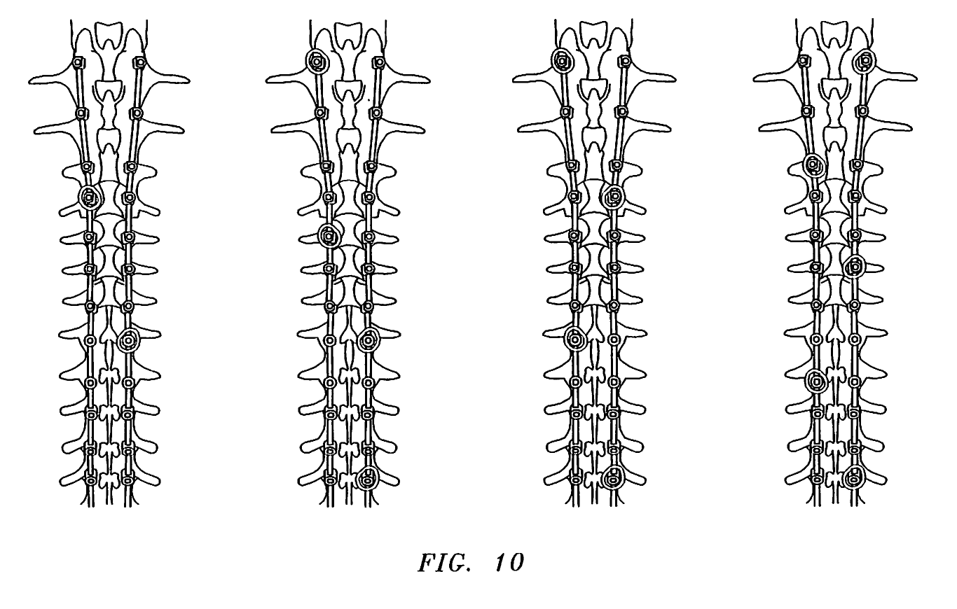

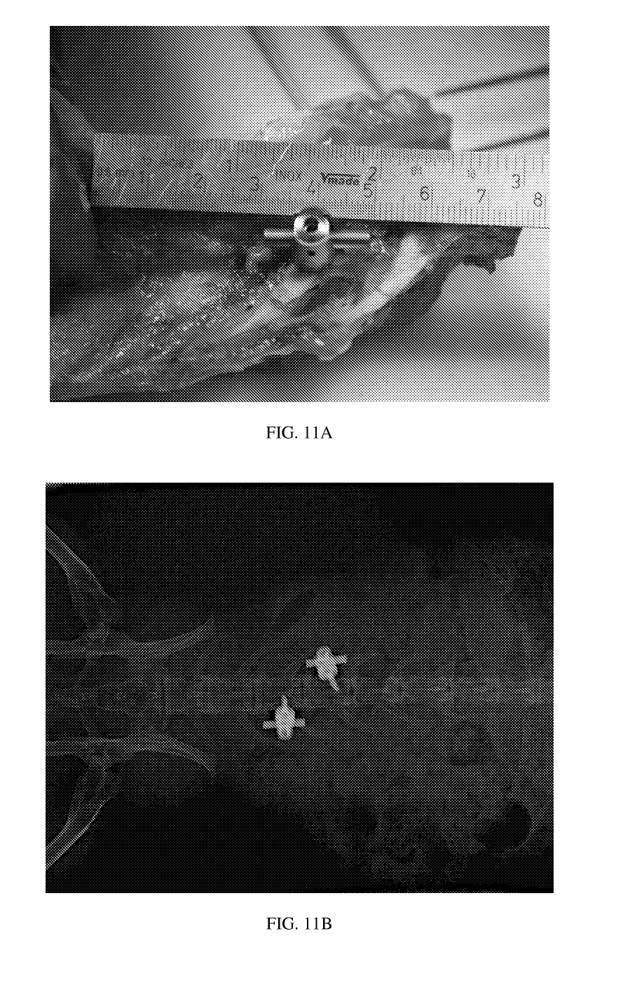

Specific embodiments and methods of using the same are described in conjunction with FIGS. 1-13 and 15-16. These figures are envisioned to help in describing the invention but are in no way meant to be limiting in scope of the invention.

FIG. 1 illustrates an implant having a drug eluting cover 05 already affixed thereto. Implant 15 can be attached to bone in the body by an attaching element 30 of the implant 15. A drug eluting cover of the present invention 10 can be covered on at least a portion of implant 15 so that one surface of the cover comes in contact with the surface 25 of the implant 15. As shown, the drug to be eluted from the cover 10 is indicated by the multiple of specks 20 dispersed throughout the cover 10. As stated, the thickness of the cover and the concentration, and type of drug, can and will vary form cover to cover but are envisioned to fall within the present invention. Also as shown, the cover is designed to be in close contact with the surface of the implant to avoid and reduce spaces and gaps. Since the cover can elute antibacterial agents and the like either towards the adjacent soft tissues as well as towards the surface of the implant, infections can be kept to minimum.

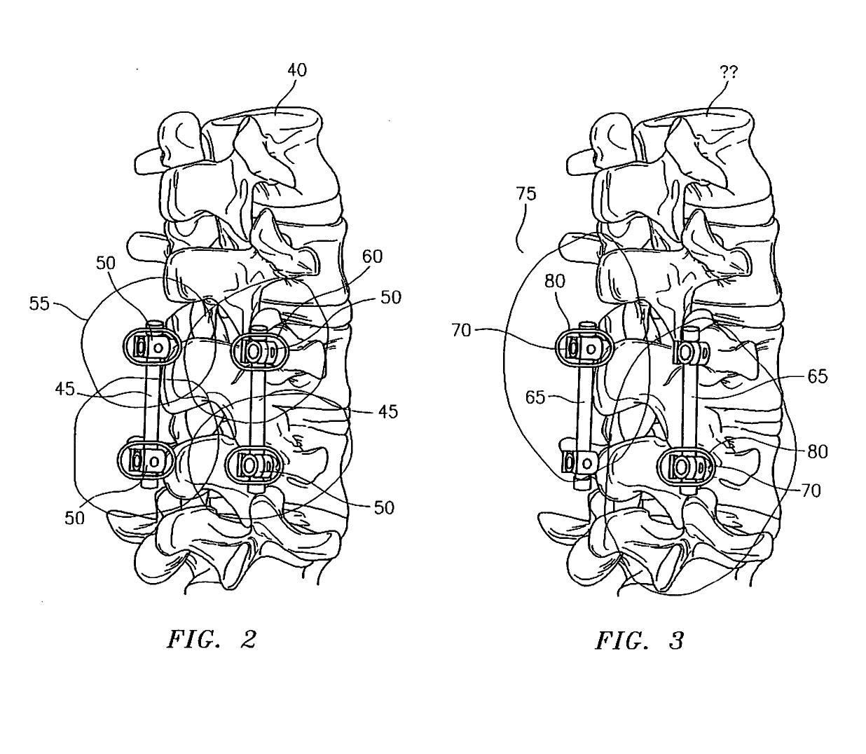

FIG. 2 illustrates a cervical column 40 having two rods 45 and four connectors 50. Each connector 50 is covered with a drug eluting cover 60 of the present invention, which has a particular concentration of drug to be eluted to the surrounding areas. This figure demonstrates a situation where the concentration of drug in the drug eluting cover 60 of the present invention is low and therefore results in a relatively small range of drug elution creating a small drug area 55. Having a small drug area 55, more drug eluting covers 60 can be used so that the estimated drug eluting areas can overlap to provide a larger total drug zone. The number of covers used and the positioning of the covers can be determined by the surgeon.

Accordingly, FIG. 3 illustrates a situation where the drug eluting covers 80 affixed on connectors 70 which are attached to rods 65 to the cervical column 80 have a higher concentration than the covers shown in FIG. 2. For this reason, only two drug eluting covers 80 are used which produce larger drug eluting areas that overlap to provide an associated drug zone.

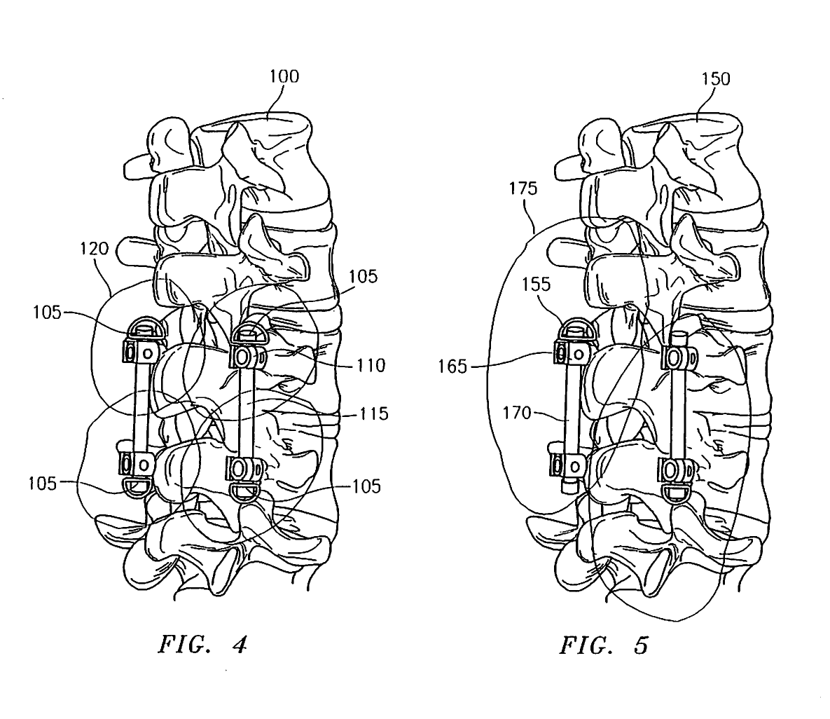

FIGS. 4 and 5 illustrate drug-eluting covers (105 in FIGS. 4 and 155 in FIG. 5) affixed to the rod-ends of the rod and connector assemblies shown attached to a cervical column. In FIG. 4 the drug concentration of the drug-eluting covers is smaller than the concentration of the covers in FIG. 5 thereby producing a smaller drug eluting range than that of the covers shown in FIG. 5. Accordingly, fewer drug-eluting covers can be used in FIG. 5 than in FIG. 4.

The number of drug-eluting covers and drug-eluting spinal implants used in a particular implant surgery as well as the concentration of drug that each drug-eluting cover has can vary according to the particular needs. Each cover used can have the same or different drug concentrations thereby producing a variety of customized drug zones as may be necessary in each implant.

FIGS. 6 through 10 show different levels and/or combination of covers that can be used to provide customized drug delivery.

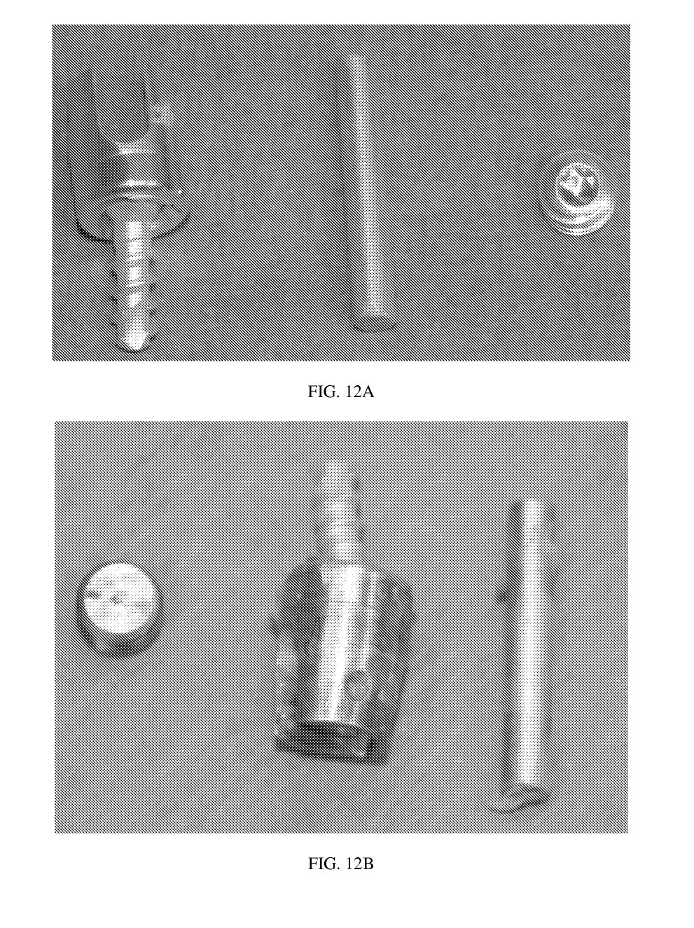

FIGS. 11a and 11b show surgical pictures of an implant positioned in a Rabbit Model used to test the efficiency of nanosilver vs. particular antibiotic combinations provided in the coating on the implants.

FIGS. 12a and 12b show surgical pictures of the implants coated with nanosilver before implantation and explants after 7 days.

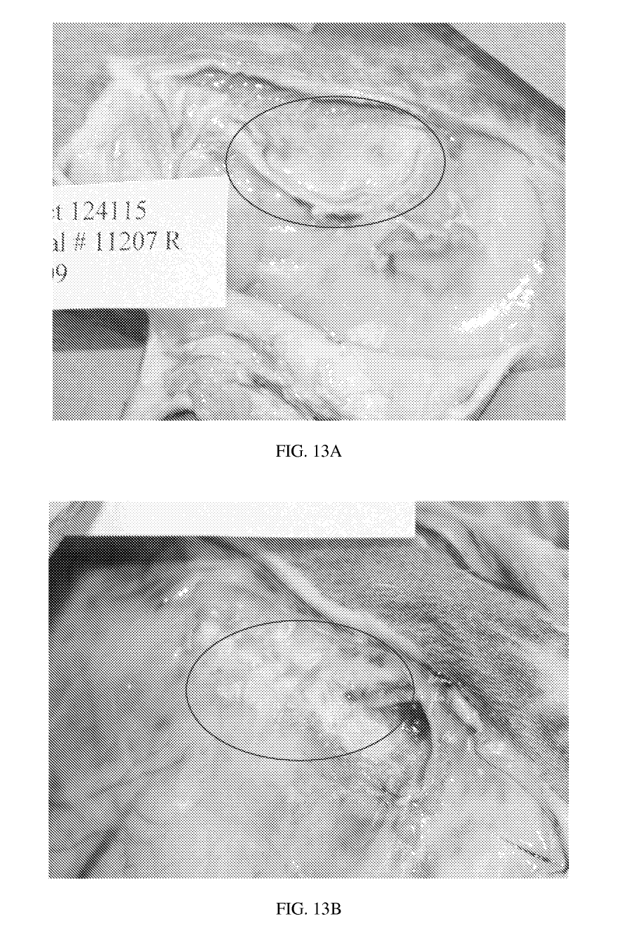

FIG. 13a shows a rabbit suture 7 days after implant of a control tissue which shows clear signs of infection. The site of infection is circled and labeled in FIG. 13a. FIG. 13b shows a clean rabbit suture line of a nanosilver coated implant also 7 days after being implanted; however, as shown in the results in Table 1, the nanosilver coating was ineffective in fighting off infection.

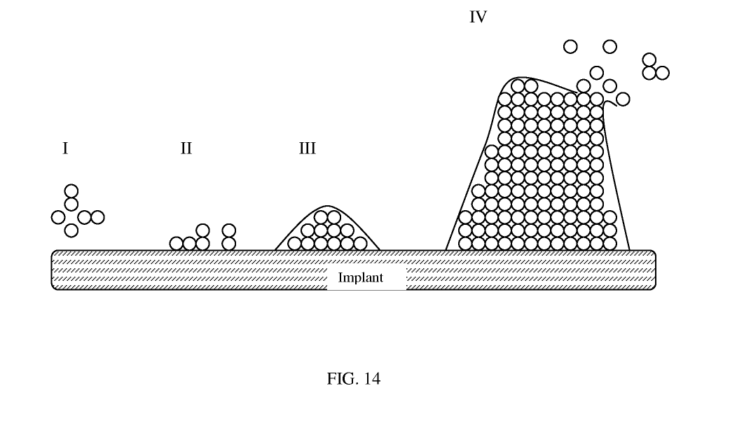

FIG. 14 shows a biofilm and microbial recovery from an explanted device via sonication of the device.

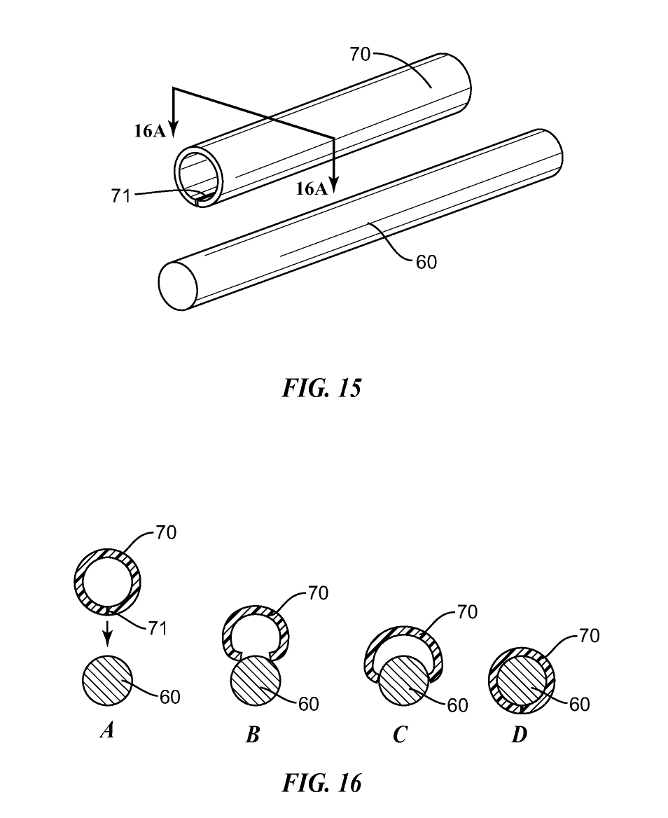

One method of affixation of these and similar-type cylindrical sleeves to a spinal rod is illustrated in FIG. 15. Preformed drug-eluting cylindrical sleeve 70 made of a silicone elastomer and possessing lengthwise slit 71 is press-fitted to spinal rod 60. 5A shows the cross-sectional line. FIG. 16 shows sequence A-D illustrating the deformation of the sleeve under continuously applied pressure until it snaps fully in place (stage D).

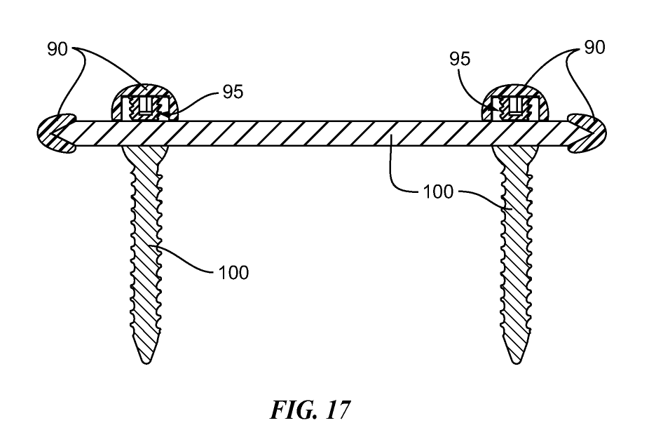

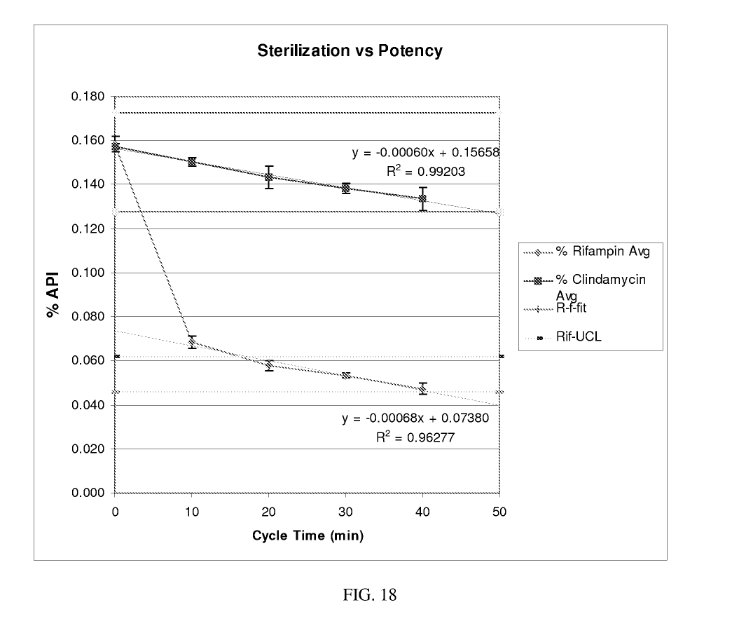

FIG. 17 shows various snap-to-fit covers in the form of a cap which are placed on different parts of an implant, such as the ends, pointed protrusions, screw heads, etc. The snap-to-fit covers not only provide a source of drugs(s), but aid in reducing irritation to adjacent tissue. As stated above, lubricious coatings can also be added to aid in reducing irritation.

The following examples are illustrative of the manufacture of the drug-eluting spinal implant of the invention.

### EXAMPLES

A cylindrical sleeve made of silicone elastomer and having an average wall thickness of 0.4 mm to 2.5 mm was used as a drug-eluting device for a steel spinal rod component of a known or conventional spinal fixation system. The sleeve contains minocycline at a loading of about 2.0 μg/mg and rifampin at a loading of about 3.0 ug/mg (about 0.2 wt % minocycline and about 0.3 wt % rifampin, see U.S. Pat. No. 4,917,686). See FIGS. 15-16 for a perspective view of a cylindrical sleeve used as a drug-eluting device for a spinal rod component.

A substantially identical cylindrical sleeve of silicone elastomer contains clindamycin at 0.15 weight percent and rifampin at 0.054 weight percent.

### Methods and Procedures

### Custom In-Life Studies: Device Infection Model Development

A clinical problem when using implants is that 3-5% of device implants develop infection. Infections often involve formation of microbial biofilms around the implant device which are very resistant to standard antibiotic therapies resulting in high morbidity and mortality of patients. Treatment often requires removal of infected device, debridement, and new implant(s) at a very high cost. Further, reimbursement for treating hospital-acquired infections is being limited.

### Spinal Device Infection Animal Model: Specific Considerations

Considerations in the design of spinal implant(s) in in-life anti-microbial tests include: studying designs which are specific to a clinical situation such as the spinal site and the combination of screw and rod to mimic geometry, using clinically relevant microbes such as S. aureus, using reproducible and quantifiable endpoints such as by using sonications of explanted screw/rod to recover adherent bacterial (“biofilm”, See FIG. 14), creating infection with dose(s) high enough to create consistent infection without mortality, but low enough to be amenable to treatment in dosing studies and considering desired claims in the study design to show reduction of infection.

### Device Infection Studies-Microbial Pathogens

Microorganism strains used in past studies include Staphylococcus aureus (MSSA, MRSA), Staphylococcus epidermidis, Staphylococcus capitis, Escherichia coli, Acinetobacter baummanii, Pseudomonas aeruginosa and P. Acnes. Each strain can be obtained from ATCC (American Type Culture Collection) or characterized clinical isolate from Sponsor. Each new organism requires In vitro characterization to determine growth curve and In vivo dosing studies to determine dose that creates consistent infection without mortality.

### Dosing Study of In Vivo Spinal Screw Infection

In the study design (final of 3 dosing studies) there are three dose groups (1×10³, 1×10², 2×10¹ CFU) including 2-3 animals per group on bilateral sites. The test implant is an uncoated spinal screw and rod which was implanted for an in-life duration of 7 days. The explant measurements include photograph documentation of the implant site, recordation of gross observations, radiographs (post-surgical and termination to confirm test implant placement), hematology and sonication/vortex of explanted screw/rod set to assess bacteria on device.

TABLE 1
Dosing Study: Device Sonication Results

| Group | | | Sonicant 1 | | | | | Sonicant 2 | | | | | | | Isolate Identification | |
|---|---|---|---|---|---|---|---|---|---|---|---|---|---|---|---|---|
| (Dose) | Animal # | Side | U | 10⁻¹ | 10⁻² | 10⁻³ | Cfu/mL | U | 10⁻¹ | 10⁻² | 10⁻³ | Cf/mL | Swab | Blood | Gram Stain | API ID |
| 1000 | 10837 | Right | TNC | TNC | TNC | 134 | 6.70E+05 | TNC | TNC | 82 | 4 | 4.10E+04 | + | − | GPC | SA |
| CFU | | Left | TNC | TNC | TNC | 49 | 2.45E+05 | TNC | TNC | 57 | 5 | 2.85E+04 | + | − | GPC | SA |
| per site | 10839 | Right | TNC | TNC | TNC | 80 | 4.00E+05 | TNC | TNC | 112 | 15 | 5.60E+04 | + | − | GPC | SA |
| | | Left | TNC | TNC | TNC | 212 | 1.06E+06 | TNC | TNC | TNC | 21 | 1.05E+05 | + | − | GPC | SA |
| | NA | | | | | | | | | | | | | | | |
| 100 | 10840 | Right | TNC | TNC | TNC | 122 | 6.10E+05 | TNC | TNC | TNC | 37 | 1.85E+05 | + | − | GPC | SA |
| CFU | | Left | TNC | TNC | TNC | TNC | TNC | TNC | TNC | TNC | 115 | 5.75E+05 | + | − | GPC | SA |
| per site | 10844 | Right | TNC | TNC | TNC | 160 | 8.00E+05 | TNC | TNC | TNC | 52 | 2.60E+05 | + | − | GPC | SA |
| | | Left | TNC | TNC | TNC | 74 | 3.70E+05 | TNC | TNC | 110 | 12 | 5.50E+04 | + | − | GPC | SA |
| | 10843 | Right | TNC | TNC | TNC | 20 | 1.00E+05 | TNC | TNC | 68 | 20 | 3.40E+04 | + | − | GPC | SA |
| | | Left | TNC | TNC | 94 | 8 | 4.70E+04 | TNC | TNC | 76 | 6 | 3.80E+05 | + | − | GPC | SA |
| 20 CFU | 10841 | Right | TNC | TNC | TNC | 106 | 5.30E+05 | TNC | TNC | 88 | 16 | 4.40E+04 | + | − | GPC | SA |
| per site | | Left | TNC | TNC | TNC | TNC | TNC | TNC | TNC | 198 | 28 | 9.90E+04 | + | − | GPC | SA |
| | 10842 | Right | TNC | TNC | TNC | 136 | 6.80E+05 | TNC | TNC | 120 | 18 | 6.00E+04 | + | − | GPC | SA |
| | | Left | 16 | 1 | 0 | 0 | 8.00E+02 | 0 | 1 | 0 | 0 | 5.00E+01 | + | − | GPC | SA |
| | 10838 | Right | 0 | 0 | 0 | 0 | 0 | 0 | 0 | 0 | 0 | 0 | + | − | GPC | SA |
| | | Left | 0 | 0 | 0 | 0 | 0 | 0 | 0 | 0 | 0 | 0 | + | − | GPC | SA |

TABLE 2
Dosing Study: Summary Table

| Group | | 1^{st} Sonication | 2nd Sonication | |
|---|---|---|---|---|
| (Target | | Recovery | Recovery | Swab |
| Dose) | Actual Dose | (ave. CFU/mL) | (ave. CFU/mL) | (+/−) |
| High Dose | 0.4-0.8 × 10³ | 5.9 × 10⁵ | 5.8 × 10⁴ | +(4/4) |
| (1 × 10³) | | (4/4 positive) | (4/4 positive) | |
| Middle Dose | 0.9-1.2 × 10² | 5.2 × 10⁵ | 1.9 × 10⁵ | +(6/6) |
| (1 × 10²) | | (6/6 positive) | (6/6 positive) | |
| Low Dose | 0.8-0.9 × 10¹ | 5.4 × 10⁵ | 5.0 × 10⁴ | +(6/6) |
| (2 × 10¹) | | (5/6 positive) | (5/6 positive) | |

Three rabbit implant studies were conducted comparing microbial growth on implants coated with nanosilver and implants coated with specific combinations of antibiotics. Although the implants used were directly coated, these results are used to show the effects of a drug-eluting cover used with implants. Below are several tables that outline 3 study groups, namely nanoSilver (Efficacy Study #1), Minocycline/Rifampin and Clindamycin/Rifampin (M/R coating and C/R coating-Efficacy Study #2).

Efficacy Study #1—Nanosilver Coating

Study Design (efficacy study, silver coatings): Three study groups include implants which are uncoated, coated with silver coating A (low levels of silver) and coated with silver coating B (high levels of silver). The Staphylococcus aureus dose was 1×10² CFU total per site. There were 3 animals per group with studies on bilateral sites. The test implant was a spinal screw with an uncoated (control) or coated rod which was implanted into the rabbit model for 7 days (see FIGS. 12a and 12b). Measurements of the explanted device include photograph documentation of the implant site (see FIGS. 13a and 13b), recordation of gross observations, radiographs [post-surgical and at termination to confirm proper test implant placement (see FIG. 11b)], hematology and sonication/vortexing (of two animals) of explanted screw/rod sets to assess bacteria on device.

TABLE 3
Sonication Results: Efficacy Study #1 - NanoSilver

| | | | Sonicant 1 | | | | | Sonicant 2 | | | | | | | Isolate Identification | |
|---|---|---|---|---|---|---|---|---|---|---|---|---|---|---|---|---|
| Group | Animal # | Side | U | 10⁻¹ | 10⁻² | 10⁻³ | CFU/mL | U | 10⁻¹ | 10⁻² | 10⁻³ | CFU/mL | Swab | Blood | Gram Stain | API ID |
| Control | 11206 | Right | TNC | TNC | TNC | 154 | 7.70E+05 | TNC | TNC | 116 | 11 | 5.80E+04 | + | − | GNC | SA |
| | | Left | TNC | TNC | TNC | 106 | 5.30E+05 | TNC | TNC | 178 | 19 | 8.90E+04 | + | − | GPC | UP |
| | 11207 | Right | TNC | TNC | TNC | 180 | 9.00E+05 | TNC | TNC | 83 | 14 | 4.15E+04 | + | − | GNC | SA |
| | | Left | TNC | TNC | 243 | 28 | 1.22E+05 | TNC | TNC | 64 | 7 | 3.20E+04 | + | − | GPC | SA |
| | 11208 | Right | TNC | TNC | TNC | 160 | 8.00E+05 | TNC | TNC | 260 | 23 | 1.30E+05 | + | − | GPC | SA |
| | | Left | TNC | TNC | TNC | 167 | 8.35E+05 | TNC | TNC | 220 | 15 | 1.10E+05 | + | − | GVC | SA |
| Coating | 11209 | Right | TNC | TNC | TNC | 143 | 7.15E+05 | TNC | TNC | TNC | 75 | 3.75E+05 | + | − | GVC | SA |
| A | | Left | TNC | TNC | 165 | 28 | 8.25E+05 | TNC | TNC | 45 | 5 | 2.25E+04 | + | − | GPC | SA |
| | 11210 | Right | TNC | TNC | TNC | 292 | 1.46E+06 | TNC | TNC | TNC | 89 | 4.45E+05 | + | − | GPC | SA |
| | | Left | TNC | TNC | TNC | 167 | 7.65E+05 | TNC | TNC | 169 | 10 | 8.45E+04 | + | − | GPC | SA |
| | 11211 | Right | TNC | TNC | TNC | 106 | 5.30E+05 | TNC | TNC | 197 | 12 | 9.85E+04 | + | − | GPC | SA |
| | | Left | TNC | TNC | TNC | TNC | TNC | TNC | TNC | TNC | 96 | 4.80E+05 | + | − | GPC | SA |
| Coating | 11200 | Right | TNC | TNC | TNC | 111 | 5.55E+05 | TNC | TNC | 265 | 29 | 1.33E+05 | + | − | GNC | SA |
| B | | Left | TNC | TNC | TNC | 133 | 6.65E+05 | TNC | TNC | TNC | 42 | 2.10E+05 | + | − | GPC | SA |
| | 11201 | Right | TNC | TNC | TNC | 240 | 1.20E+05 | TNC | TNC | TNC | 44 | 2.20E+05 | + | − | GVC | SA |
| | | Left | TNC | TNC | TNC | 162 | 8.10E+05 | TNC | TNC | TNC | 49 | 2.45E+05 | + | − | GVC | SA |
| | 11202 | Right | TNC | TNC | TNC | 39 | 1.95E+05 | TNC | TNC | 242 | 26 | 1.21E+05 | + | − | GVC | SA |
| | | Left | TNC | TNC | TNC | 72 | 3.60E+05 | TNC | TNC | 236 | 22 | 1.18E+05 | + | − | GPC | SA |

TABLE 4
Efficacy Study #1 (Ag): Summary Table

| | Actual Dose | | | |
|---|---|---|---|---|
| Group | (Target Dose = | 1^{st} Sonication | 2^{nd} Sonication | |
| (Target | 1 × 10² | Recovery | Recovery | Swab |
| Dose) | Total CFU) | (ave. CFU/mL) | (ave. CFU/mL) | (+/−) |
| Control | 0.9 × 10² | 6.5 × 10⁵ | 7.7 × 10⁴ | +(6/6) |
| (No | | (6/6 positive) | (6/6 positive) | |
| Coating) | | | | |
| Low | 0.9 × 10² | 9.7 × 10⁵ | 2.8 × 10⁵ | +(6/6) |
| Coating | | (6/6 positive) | (6/6 positive) | |
| High | 0.9 × 10² | 6.3 × 10⁵ | 1.4 × 10⁵ | +(6/6) |
| Coating | | (5/6 positive) | (5/6 positive) | |

As can be seen from the Summary Tables 2 and 4, high, middle, or low dosage levels of silver were ineffective in reducing the average CFU/ml in 1^{st} and 2^{nd} Sonication Recovery. Thus, it can be concluded that silver is ineffective in preventing/reducing infection in the pocket of the implant.

Efficacy Study #2—Minocycline/Rifampin and Clindamycin/Rifampin

Study Design (efficacy study, antibiotic coatings): Three study groups include implants which were uncoated, coated with Minocycline/Rifampin (M/R coating) or coated with Clindamycin/Rifampin (C/R coating) snap-to-fit implant covers. The Staphylococcus aureus dose was 1×10² CFU total per site. There were three animals per group with studies conducted on bilateral sites. The test implant was a spinal screw with an uncoated (control) rod or a rod covered with the snap-to-fit cover which was implanted into the rabbit model for 7 days (see FIGS. 12a and 12b).

The Minocycline/Rifampin coating was made with 2.046 μg/mg of minocycline and 2.977 μg/mg of rifampin imbedded in a 0.015 inch or 0.4 mm thick silicone tube that was 2 cm long that weighed 93.6 mg for total drug on the rod of 191 μg minocycline and 278 μg of rifampin ((0.191 mg minocycline÷93.6 mg of silicone)×100=about 0.2% by weight). The Minocycline/Clindamycin coating was made with 0.15 wt % Clindamycin and 0.054 wt % rifampin imbedded in a silicone tube that weighed 174.2 mg for total drug on the rod of 261 μg clindamycin and 94 μg of rifampin ((0.094 mg lindamycin÷174.2 mg of silicone)×100=about 0.054% by weight).

Measurements of the explanted device include photograph documentation of the implant site (see FIGS. 13a and 13b), recordation of gross observations, radiographs were conducted post-surgical and at termination to confirm proper test implant placement (see FIG. 11b), hematology and sonication/vortexing (of three animals) of explanted screw/rod set to assess bacteria on device.

TABLE 5
Sonication Results: Efficacy Study #2

| | | | Sonicant 1 | | | | | Sonicant 2 | | | | | | | Isolate Identification | |
|---|---|---|---|---|---|---|---|---|---|---|---|---|---|---|---|---|
| Group | Animal # | Side | U | 10⁻¹ | 10⁻² | 10⁻³ | CFU/mL | U | 10⁻¹ | 10⁻² | 10⁻³ | CFU/mL | Swab | Blood | Gram Stain | API ID |
| Control | 11727 | Right | TNC | TNC | TNC | 118 | 5.90E+05 | TNC | TNC | 142 | 17 | 7.10E+04 | + | − | TBD | TBD |
| (No Tx) | | Left | TNC | TNC | TNC | 239 | 1.20E+06 | TNC | TNC | 215 | 17 | 1.08E+05 | + | − | TBD | TBD |
| | 11728 | Right | TNC | TNC | TNC | 180 | 9.00E+05 | TNC | TNC | 83 | 14 | 4.15E+04 | + | − | TBD | TBD |
| | | Left | TNC | TNC | 243 | 28 | 1.22E+05 | TNC | TNC | 64 | 7 | 3.20E+04 | + | − | TBD | TBD |
| | 11728 | Right | | | | | | | | 13 | 1 | 8.80E+03 | NA | NA | NA | NA |
| | Sonicant | Left | | | | | | | | 31 | 2 | 1.55E+04 | NA | NA | NA | NA |
| | 3: | | | | | | | | | | | | | | | |
| M/R Tx | 11721 | Right | 0 | 0 | 0 | 0 | 0 | 0 | 0 | 0 | 0 | 0 | − | − | NA | NA |
| | | Left | 0 | 0 | 0 | 0 | 0 | 0 | 0 | 0 | 0 | 0 | − | − | NA | NA |
| | 11722 | Right | 0 | 0 | 0 | 0 | 0 | 0 | 0 | 0 | 0 | 0 | − | − | NA | NA |
| | | Left | 0 | 0 | 0 | 0 | 0 | 0 | 0 | 0 | 0 | 0 | − | − | NA | NA |
| | 11723 | Right | 0 | 0 | 0 | 0 | 0 | 0 | 0 | 0 | 0 | 0 | − | − | NA | NA |
| | | Left | 0 | 0 | 0 | 0 | 0 | 0 | 0 | 0 | 0 | 0 | − | − | NA | NA |
| C/R Tx | 11724 | Right | 0 | 0 | 0 | 0 | 0 | 0 | 0 | 0 | 0 | 0 | − | − | NA | NA |
| | | Left | 0 | 0 | 0 | 0 | 0 | 0 | 0 | 0 | 0 | 0 | − | − | NA | NA |
| | 11725 | Right | 0 | 0 | 0 | 0 | 0 | 0 | 0 | 0 | 0 | 0 | − | − | NA | NA |
| | | Left | 0 | 0 | 0 | 0 | 0 | 0 | 0 | 0 | 0 | 0 | − | − | NA | NA |
| | 11726 | Right | 0 | 0 | 0 | 0 | 0 | 0 | 0 | 0 | 0 | 0 | − | − | NA | NA |
| | | Left | 0 | 0 | 0 | 0 | 0 | 0 | 0 | 0 | 0 | 0 | − | − | NA | NA |

TABLE 6
Summary Table

| | Actual Dose | 1^{st} Sonication | 2nd Sonication | 3rd Sonication | Swab |
|---|---|---|---|---|---|
| | (Target Dose = | Recovery | Recovery | Recovery | (positive/ |
| Group | 1 × 10² Total CFU) | (ave. CFU/mL) | (ave. CFU/mL) | (ave. CFU/mL) | total) |
| Control | 1.0-1.3 × 10² | 5.6 × 10⁵ | 7.2 × 10⁴ | 1.9 × 10⁴ | 4/4 |
| (No | | (4/4 positive) | (4/4 positive) | (4/4 positive) | |
| Treatment) | | | | | |
| M/R | 1.0-1.3 × 10² | 0 | 0 | 0 | 0/6 |
| Treatment | | (0/6 positive) | (0/6 positive) | (0/6 positive) | |
| C/R | 1.0-1.3 × 10² | 0 | 0 | 0 | 0/6 |
| Treatment | | (0/6 positive) | (0/6 positive) | (0/6 positive) | |

In summary, it can be seen from the studies conducted that a consistent Staphylococcus aureus infection can be created at the implant site. In efficacy study # 1 nanosilver coatings were shown to be ineffective at reducing infection in the pocket and on the device in this infection model. Efficacy study #2 shows a snap-to-fit implant cover containing clindamycin/rifampin and/or minocycline/rifampin using a silicone sleeve to cover the rods was effective at eliminating infection in the pocket and on the device in this infection model. Thus, antibiotic infused covers of the present invention are effective in eliminating infection in the pocket and on the implant. In particular, drug-elutable implant covers containing clindamycin/rifampin and/or minocycline/rifampin.

While the invention has been illustrated and described in detail the drawings and foregoing description, the same is considered to be illustrative and not restrictive in character, it is understood that only the preferred embodiments have been shown and described and that all changes and modifications that come within the spirit of the invention are desired to be protected. All patent applications, patents and all documents cited herein are hereby incorporated by reference in their entirety.

What is claimed is:

1. A drug-eluting implant cover comprising:
a pre-formed snap-to-fit implant cover fabricated from a drug-eluting biocompatible matrix comprising at least one elutable drug wherein said drug-eluting biocompatible matrix is configured for therapeutically delivering said at least one elutable drug to a surgical area and to facilitate non-irritating motion across adjacent tissue.

2. The drug-eluting implant cover of claim 1 wherein said drug-eluting biocompatible matrix is made of or coated with a friction-reducing material in order to reduce irritation of adjacent tissue with motion across said tissue.

3. The drug-eluting implant cover of claim 2 wherein said friction-reducing material is selected from the group consisting of silicone, hydrogel, xerogel, polyethylene, lotions, lubricants, oils, greases and combinations thereof so as to facilitate non-irritating motion across soft tissue.

4. The drug-eluting implant cover of claim 3, wherein said hydrogel is selected from the group consisting of a polyvinyl alcohol, a polyacrylic acid, a polyarylamide, a poly(acrylonitrile-acrylic acid), a polyurethane, a polyethylene glycol, a poly(N-vinyl-2-pyrrolidone), a gelatin, a collagen, a polysaccharide, a cellulose, and combinations thereof.

5. The drug-eluting implant cover of claim 1, wherein said drug-eluting biocompatible matrix comprises a film-forming polymer.

6. The drug-eluting implant cover of claim 5, wherein said film-forming polymer is selected from the group consisting of bioresorbable polymer, hydrogel, polylactide, polyglycolide, copolymers of polylactide, polyglycolide, polycaprolactone, polyorthoester, silicone, polyurethane, silicone-polyurethane copolymers, polyethylene, polypropylene, polyester, polyaryletherketone, polyimide, polyetherimide, polyamide, polysulfone and combinations thereof.

7. The drug-eluting implant cover of claim 6, wherein said hydrogel is selected from the group consisting of a polyvinyl alcohol, a polyacrylic acid, a polyarylamide, a poly(acrylonitrile-acrylic acid), a polyurethane, a polyethylene glycol, a poly(N-vinyl-2-pyrrolidone), a gelatin, a collagen, a polysaccharide, a cellulose, and combinations thereof.

8. The drug-eluting implant cover of claim 1, wherein said elutable drug is selected from the group consisting of at least one antibiotic agent, antiseptic agent, analgesic, bone growth promoting substance, anti-inflammatant, anti-arrhythmics, anti-coagulants, antifungal agent, growth inhibitors, growth stimulators, steroid, anti-adhesion agent, growth factor, wound-healing accelerator, immuno-suppressant, bone morphogenic protein, soft tissue growth inhibitors and combinations thereof.

9. The drug-eluting implant cover of claim 1, wherein the elutable drug is rifampin in combination with minocycline and/or clindamycin.

10. The drug-eluting implant cover of claim 9, wherein rifampin is present in an amount of between about 0.03 wt % and about 0.07 wt % and clindamycin is present in an amount of between about 0.1 wt % and about 0.2 wt %.

11. The drug-eluting implant cover of claim 10, wherein rifampin is present at between about 0.05 wt % and about 0.06 wt % and clindamycin is present between about 0.1 wt % and about 0.2 wt %.

12. The drug-eluting implant cover of claim 10, wherein the drug-eluting biocompatible matrix comprises a silicone elastomer.

13. The drug-eluting device of claim 9, wherein minocycline is present in an amount between about 0.02 wt % and about 0.8 wt % and rifampin is present in an amount between about 0.03 wt % and about 1.0 wt %.

14. The drug-eluting device of claim 9, wherein minocycline is present in an amount between about 0.1 wt % and about 0.3 wt % and rifampin is present in an amount between about 0.1 wt % and about 0.4 wt %.

15. The drug-eluting device of claim 13, wherein the biocompatible matrix comprises a silicone elastomer.

16. The drug-eluting implant cover of claim 1, wherein said implant cover is configured to fit an implant selected from the group consisting of a spinal stabilization implant, spinal dynamic implant, spinal rod, spinal plate, anterior spinal plate, spinal rod component of a spinal fixation system, spinal fixation system, spinal interbody fusion device, bone screw, pedicle screw, crosslink component, spinal hook, interspinous process spacer, bone rod and sub-assembly components thereof.

17. The drug-eluting implant cover of claim 1, wherein said drug-eluting biocompatible matrix has a thickness of between about 0.1 mm and about 5 mm.

18. A drug-eluting implant cover kit comprising at least one snap-to-fit implant cover of claim 1 in a sterile container.

19. The drug-eluting implant cover kit of claim 18, wherein said at least one elutable drug comprises a combination of minocycline and rifampin or a combination of clindamycin and rifampin.

20. The drug-eluting implant cover kit of claim 19, wherein the elutable drug comprises a combination of clindamycin and rifampin and rifampin is present at between about 0.05 wt % and about 0.06 wt % and clindamycin is present between about 0.1 wt % and about 0.2 wt % and the drug-eluting biocompatible matrix comprises a silicone elastomer.
